(19) United States
(12) Patent Application Publication
(10) Pub. No.: US 2026/0354681 A1
(43) Pub. Date: Nov. 12, 2026

(54) DEVICE FOR PERFORMING GONIOSCOPY

(71) Applicant: Heidelberg Engineering GmbH, Heidelberg (DE)

(72) Inventors: Michael STENDER, Lübeck (DE); Andreas FRITZ-BOUTELEUX, Lübeck (DE)

(73) Assignee: Heidelberg Engineering GmbH, Heidelberg (DE)

(21) Appl. No.: 19/117,117

(22) PCT Filed: Jul. 7, 2023
(86) PCT No.: PCT/EP2023/068814

§ 371 Date: Mar. 31, 2025

(30) Foreign Application Priority Data
Sep. 30, 2022 (DE) 10 2022 125 337.6

(51) Int. Cl. A61B 3/117; A61B 3/00; A61B 3/10

(52) U.S. Cl. CPC A61B 3/117; A61B 3/0025; A61B 3/102; A61B 2505/05

(57) ABSTRACT

The invention relates to a device (13) for performing gonioscopy, by means of which device a three-dimensional volume (14) of a sample can be captured by means of optical coherence tomography as a raw dataset. With respect to the problem of overcoming the technical disadvantages of a gonioscopic lens, the device is characterised in that a visualising device (15) is provided, by means of which a virtual gonioscopy view (15a) can be generated from the raw dataset, which view appears to be taken from a virtual camera plane (16) in the interior of the sample. The invention also relates to a method for capturing the structures of the iridocorneal angle (14).

The invention relates to a device according to the preamble of claim 1.

The term optical coherence tomography (typically abbreviated as OCT) is understood as an imaging method. Two-and three-dimensional images can be obtained from light-scattering structures using this method. In this method, light having a certain bandwidth is typically split in a beam splitter into two partial beams. The first partial beam is incident on the sample to be examined or the object, the second partial beam passes through a reference section. The light reflected from the sample or the object interferes with the reference beam. The sample can be examined with depth resolution by signals from the interference, thus in the depth of the optical axis of the first partial beam, by so-called A-scans. In addition, it is possible to also scan the sample flatly or laterally using the first partial beam in order to obtain OCT images.

Against this background, in the human eye, the cornea and the iris form a formation in the anterior chamber of the eye. This formation is referred to as the iridocorneal angle. Aqueous humor can flow out through the iridocorneal angle. In the event of a pathological change of the iridocorneal angle, an increase of the internal pressure of the eye and the formation of a glaucoma can occur. The so-called Schlemm's canal extends in a ring shape in the scleral component of the iridocorneal angle, thus in the area of the sclera, and forms a collecting tube, namely a drain for the aqueous humor.

The structures in the iridocorneal angle of an eye cannot be seen directly without technical aids because of the refraction relationships at the cornea and the non-transparency of the sclera for visible light. In order to visualize these structures, a gonioscopic lens is currently used, which directly contacts the cornea, in order to bypass the refraction at the front side thereof and look “around the corner” into the iridocorneal angle with the aid of a mirror system. Operations are exclusively carried out with the aid of these lenses.

The use of a gonioscopic lens and visualization through the gonioscopic lens restricts an operating person in manifold ways. The handling of the lens and the interpretation of the visualization of the structures is difficult to learn in principle. The operating person has to guide the lens directly using a hand during the operation. This restricts the person in so far as they cannot use this hand for other purposes, for example they cannot guide a second instrument or control the visualization in parallel.

The operating person sees only the surface of the tissue. Information from the depth of the tissue is concealed to them. In addition, the external instrument used during the operation can impair the field of view of the structures in the iridocorneal angle.

This can be attributed to the flat viewing angle due to the deflection of a contact lens. However, glaucoma operations are supposed to be able to be carried out without a contact lens. There is therefore a need to improve the visualization of the structures in the iridocorneal angle area during an operation.

The invention is therefore based on the object of overcoming the technical disadvantages of a gonioscopic lens.

The present invention achieves the above-mentioned object by way of the features of claim 1.

It has first been recognized according to the invention that structures of the iridocorneal angle of the human eye are to be captured optically as well as possible. Furthermore, it has been recognized that there is a need to improve the visualization of the structures in the iridocorneal angle area during an operation; specifically, it has been recognized that this is possible by visualizing Schlemm's canal or other structures, in particular in the depth. Furthermore, it has been recognized that this visualization of an operation should not be impaired by instruments for the operation. It has been recognized according to the invention that an OCT device can be used to carry out gonioscopy if it captures a three-dimensional volume, namely an OCT volume, of a sample by means of optical coherence tomography as a raw data set and has a visualization unit, using which a virtual gonioscopy view can be generated from the raw data set, which appears to be recorded from a virtual camera plane in the interior of the sample. A refractive lens is not required for the operation using the virtual gonioscopy described here. The surgeon thus additionally has a free hand available.

The visualization unit could render a recording of the three-dimensional volume, namely treat this as a raw data set and generate the virtual gonioscopy view from the virtual camera plane from this raw data set. OCT volumes can thus be recorded intra-operatively over the circular relevant angles in the limbus, namely the transition zone between cornea and sclera, in close chronological sequence. The OCT volumes are then rendered on a virtual camera in the interior of the anterior chamber such that this full face view is similar to the view through a gonioscopic lens. The disadvantages described at the outset in glaucoma-surgery interventions can thus be eliminated.

The operating person is made capable of operating on the basis of a virtual gonioscopic OCT image. In addition, data or structures from the depth of the tissue, such as the extension of Schlemm's canal, which is concealed by the trabecular meshwork, a tissue in the anterior eye chamber, can be visualized by this rendered virtual gonioscopy. In particular, this is a decisive advantage for the surgeon, because Schlemm's canal is the destination for implants.

A real camera plane and the virtual camera plane could enclose an angle from the range 80° to 140°; preferably, the two camera planes could be oriented orthogonally to one another. A quantitative measurement of the extension of anatomical structures and also the visualization of structures which are actually concealed in the projected light image, such as Schlemm's canal for example, are enabled.

Using the detection unit, at least one volume or OCT volume could be recordable along an arc section, preferably over an angle range of 10° to 120°. OCT volumes are advantageously recorded such that the limbus of the eye having the structures necessary for the glaucoma-surgery interventions is located in the volume. It is advantageous if the OCT volume comprises at least 10° to 120° of the circular angle of the limbus, since this corresponds to the preferred viewing angles in gonioscopy.

Against this background, multiple volumes or OCT volumes could be able to be recorded in chronological succession and able to be captured as raw data sets. To ensure a sufficiently high lateral resolution with sufficiently high image repetition rate, it is advantageous to visualize only a small area around the angle ranges of interest in a small volume in the limbus.

The above-mentioned arc section could be part of a ring segment or part of a spiral, in particular an elliptical spiral. It is technically advantageous to scan ring segments around the limbus-if the angle range to be viewed has to be large- or elliptical spirals for smaller angle ranges.

An external instrument for operative treatment of the sample could be representable as transparent in the virtual gonioscopy view or optically removable therefrom by means of the visualization unit. Due to the steep angle of the OCT imaging in comparison to that of the gonioscope, shading of the view of the iridocorneal angle by external instruments used is precluded. It is thus possible to calculate an instrument used for the operation out of the gonioscopy view or show it semitransparent, since the view from the anterior chamber is solely virtual. This is not possible in classic gonioscopy.

A tracking unit could be provided, using which the light for carrying out the optical coherence tomography is trackable to a volume of a movement of the sample. If the OCT volumes, as described above, are scanned in a small area around the limbus, it is advantageous due to the movements of the eye relative to a microscope to track the movement and to track the scans accordingly between the volumes. The information about the tracking can be generated either from the OCT data themselves or by an additional camera, for example the microscope itself.

In addition, tracking of the relevant limbus region by another imaging modality is advantageous. The tracking controls the lateral placement of the OCT volume. It is advantageous to use operation microscope images for the tracking.

An adjustment unit could be provided, using which the virtual camera plane is determinable and definable automatically on the basis of structures of the sample. Alternatively or additionally, the adjustment unit could be manually operable such that the position and orientation of the virtual camera plane are settable in a user-controlled manner. The perspective of the virtual view is preferably automatically defined, namely on the basis of the segmentation of the structures of the eye in the OCT data-for example the corneal apex and the iris plane. The automatically adopted perspective of the view which corresponds to the virtual camera plane can be adapted by the operating person. For this purpose, the elevation of the camera plane or else the rotation of the plane around the optical axis of the eye can be controlled. Classic gonioscopy has to be trained. Using the device described here, the correct view is adopted automatically.

The volumes could be recordable at a repetition rate from the range 1 Hz to 1 kHz. Alternatively or additionally, the latency with which the virtual gonioscopy views are displayable could be less than 500 ms. Structures of the iridocorneal angle can be captured sufficiently well by the selection of this frequency range. It is particularly advantageous if the OCT volumes are recorded at a repetition rate of at least 20 Hz. It is also advantageous if the latency with which the rendered virtual gonioscopy views are displayed is less than 100 ms. An operating person can thus capture the actually prevailing conditions well.

A method for carrying out virtual gonioscopy, in which a gonioscopic lens is not used, but rather a device for performing optical coherence tomography, in particular of the type described here, comprises the following steps:
capturing a three-dimensional volume of an eye by means of optical coherence tomography as a raw data set from a real camera plane, in that the eye is illuminated therefrom,
generating a virtual gonioscopy view from the raw data set, wherein the virtual gonioscopy view takes place from a virtual camera plane, which is located in the eye and permits a top view of structures of the eye,
displaying structures of the iridocorneal angle of the eye in the virtual gonioscopy view.

A gonioscopic lens can be omitted due to such a method, which is executed by the device described here.

In the meaning of the above statements, a volume or OCT volume is not to be selected as unnecessarily large, because then high repetition rates cannot be achieved under the conditions of dense scanning and extension. Each OCT volume thus recorded is projected on a virtual camera plane. The projection is to take place such that its image is similar to the view through a gonioscopic lens.

The projection is to take place here such that the scleral band, the ciliary body, and the trabecular meshwork are distinguishable with the highest possible contrast.

Against this background, the mentioned structures could comprise Schlemm's canal, the ciliary band, the scleral band, the trabecular meshwork, and/or Schwalbe's line. One clinical target value for many glaucoma-surgery interventions is Schlemm's canal. Because this channel is not visible in a microscopic image, surgeons orient themselves on the pigmented trabecular meshwork, behind which Schlemm's canal is located. Schlemm's canal can be visualized or segmented using an OCT device and can accordingly be displayed highlighted.

The recording of the volume could take place over an angle range of 10° to 120° along the course of the limbus. The scanning patterns for the volumes are to be designed so that the smallest distance of the OCT voxels in the orthogonal direction to the limbus is sufficiently small that the transition of the structures ciliary band, scleral band, Schwalbe's line-is scanned sufficiently high. The above requirements make it obvious that the use of rapid OCT is advantageous.

A volume could be projected onto the virtual camera plane such that the projected image is similar to a view through a gonioscopic lens and thus forms the virtual gonioscopy view. The operating person can thus make use of familiar depictions, which are previously known to them from practice.

Various methods are conceivable for the projection of the relevant structures from the OCT volume. A simple pinhole camera model can be used as the model for the virtual camera. The OCT volume is mapped here on the displayed pixels of the virtual camera using the central projection. The camera center can be located here in the finite or infinite.

A segmentation of the transition from the anterior chamber into the fixed structures iris, ciliary body, sclera, trabecular meshwork, and cornea is advantageously provided. Using a segmentation line, a full-face projection can be calculated from a thin part of a boundary layer on the virtual camera plane with the aid of the central projection.

Another procedure provides ray casting originating from the pixels of the virtual camera plane. For this purpose, an opacity is assigned to the voxels of the OCT volume using a suitable opacity function as a function of their intensity. The opacity function is advantageously selected such that “aqueous humor” voxels are not assigned any opacity and any tissue is assigned the maximum opacity. For a good quality of this depiction, a high sensitivity of the OCT device is advantageous. The most transparent tissue after the aqueous humor is the cornea. The contrast of this to the aqueous humor should be sufficiently large. The projection via the ray casting takes place such that for each pixel of the virtual camera, the beam assigned according to the central projection through the OCT volume is calculated. Intensities of the OCT voxels are summed weighted according to their opacity. Proceeding from the camera plane, however, only a sufficient number of intensities of the voxels are summed in the beam direction until a summed opacity value exceeds a threshold value.

The positioning and alignment of the virtual camera plane are to be automatically preselected. However, the user is to be able to change the positioning and the alignment over a few degrees of freedom.

The method described here could be carried out intraoperatively, in particular during glaucoma-surgery interventions.

The method will be explained hereinafter on the basis of optional refinements:

The structures of the iridocorneal angle in the eye important for diagnosis and treatment are not visible in an operation microscope without technical aids.

Using the method described here, the structures in the iridocorneal angle can be viewed during the use of an operation microscope with the aid of intraoperative OCTs. Further instruments are not necessary

In order to be able to see the structures necessary for diagnosis or else surgical treatment, namely scleral spur, ciliary body, trabecular meshwork, Schlemm's canal, etc., volumetric tomographic recordings are created in the information-bearing regions in the limbus of the eye.

These tomographs have to be treated further in order to be able to be used for the above-described applications.

For this purpose, a virtual camera view is generated from these tomographic data for visualization such that the mentioned structures are visible.

This virtual gonioscopy view is advantageously oriented to the views familiar to surgeons, which are created by a gonioscopic lens.

For this purpose, in a corresponding pose, a virtual camera plane is placed in the anterior chamber and the tomographic data are advantageously projected onto this plane such that the above-described anatomic structures are displayed at high contrast.

It is also advantageous to display important structures from the interior of the eye tissue, such as Schlemm's canal, as the frequent target variable for surgical interventions, in this projection. The information or structures can be obtained from the tomographic recordings, in contrast to the conventional methods.

Important structures, such as the scleral spur, could advantageously be displayed highlighted by an automatic segmentation. The pose and positioning of the virtual camera plane are advantageously automatically adopted.

A good configuration results from the automatic segmentation of the anatomical structures of the eye posterior cornea, scleral spur, and iris front side. These items of information or structures are advantageously generated from the tomographic OCT data and the images of the operation microscope.

A surgeon is to have the option to control the configuration of the camera plane. The degrees of freedom for the control are therefore advantageously preconfigured and are then operable, for example, via a foot pedal, a 3D mouse, or a touch panel.

The rotational angles of the camera normals are advantageously controllable using the iris plane, as is the distance of the camera plane from the scleral spur band, the rotation of the camera plane parallel to the limbus, and/or the width and height of the camera plane.

An optional virtual camera plane in a tomographic data set transformed into cylinder coordinates is also advantageous, in which the cylinder axis points in a direction similar to the optical axis of the eye. This is equivalent to a correspondingly placed and shaped cylindrical camera surface.

Typical display screens or binoculars can be used to visualize the views of the virtual camera.

A mutual augmented reality visualization of structures from the tomographic OCT data and the operation camera images is advantageously provided.

For example, the projection of the selected virtual camera plane could be visualized in the operation microscope image.

In addition, the currently projected visible range of the iridocorneal angle could be visualized in a camera image.

A visualization of the segmented scleral spur in the virtual views is also advantageous.

A view of the projection of Schlemm's canal is also advantageous.

Static images could be visualized for the assessments of the iridocorneal angle structures before or after a surgical intervention.

Images could be displayed with a repetition rate for the surgical intervention itself or to assess dynamic processes. In the best case, this repetition rate is the conventional video rate.

Conditions for this are a chronologically short recording duration with high spatial scanning density at the same time in order to be able to see the iridocorneal angle structures with sufficiently high resolution.

In order to implement this with the current technical possibilities, a small volume in the corresponding limbal area is to be recorded.

An OCT device having a high equivalent A-scan rate is advantageous. In an embodiment having a scanning system, a high speed of the scanning movement is advantageous.

An evaluation of the relevant scanning range is advantageous for the control of the OCT volume. For example, the limbus could be segmented in the selected angles from the operation camera images and the scanner of the OCT system could be controlled from the information of a registration of the modalities such that only the smallest possible volume area is passed over in sufficiently high scanning density.

In the figures

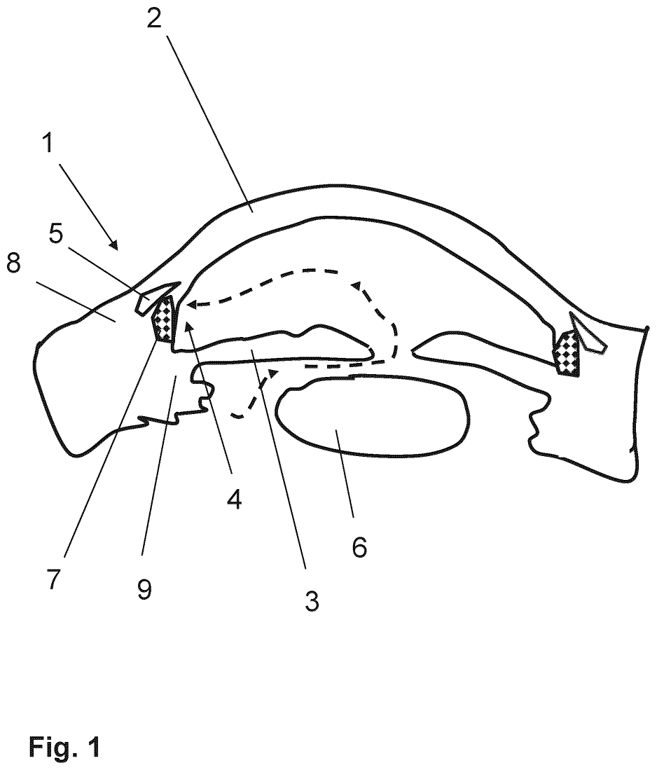

FIG. 1 shows a detailed sectional view of a human eye,

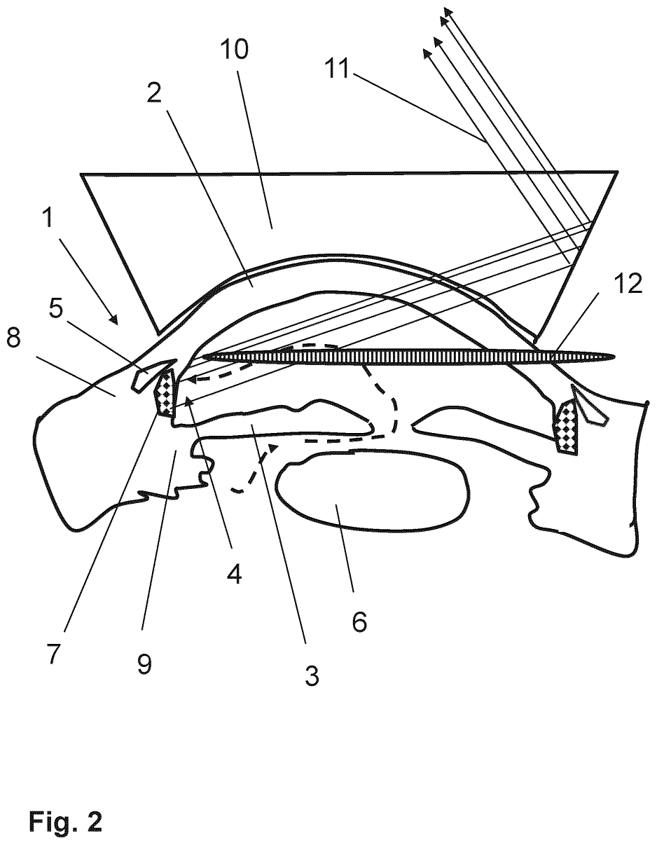

FIG. 2 shows a detailed sectional view of a human eye, on which a classic gonioscopic lens is placed, wherein the beam path through the lens is shown and wherein an external instrument throws a shadow on the actual iridocorneal angle to be examined,

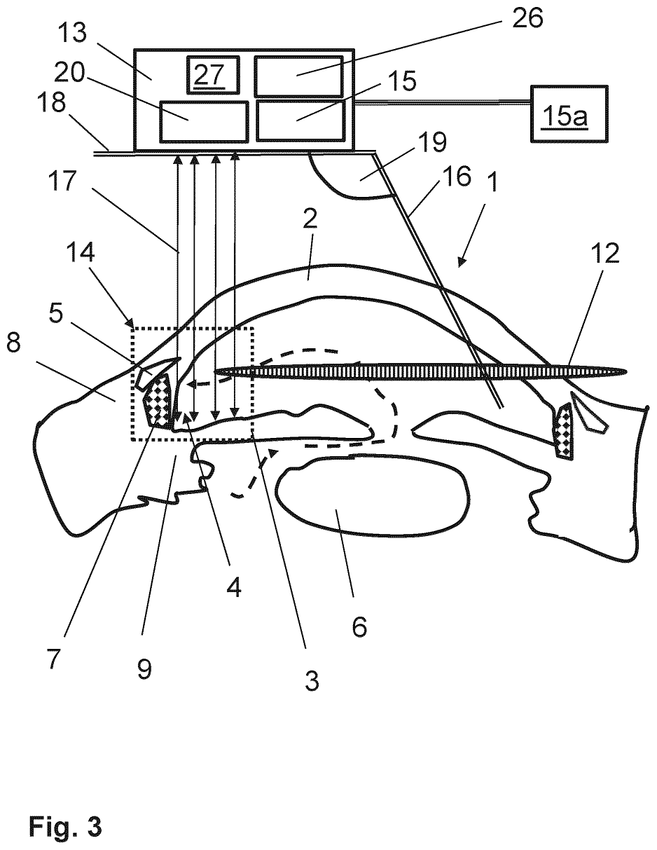

FIG. 3 shows a detailed sectional view of a human eye, into which light of an OCT device penetrates, in order to capture the iridocorneal angle from a virtual camera plane, namely a section through the structures in the iridocorneal angle and the virtual camera, wherein it is shown that an external instrument throws a shadow onto the iris, which is not relevant for an operation,

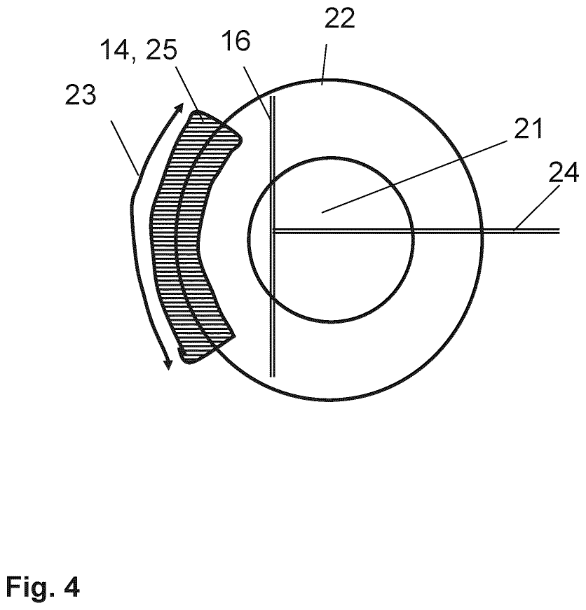

FIG. 4 shows a top view of the structures of the anterior eye section, wherein the projection of a possible target volume and a possible selection of the camera plane are shown, and

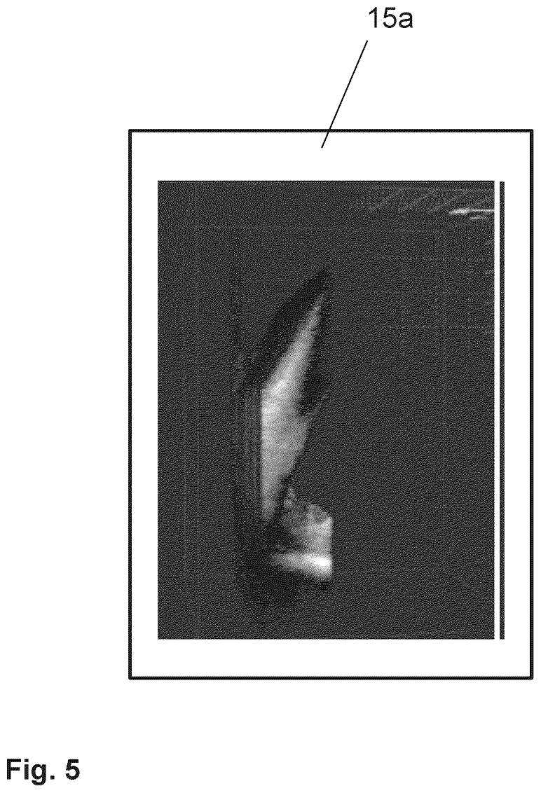

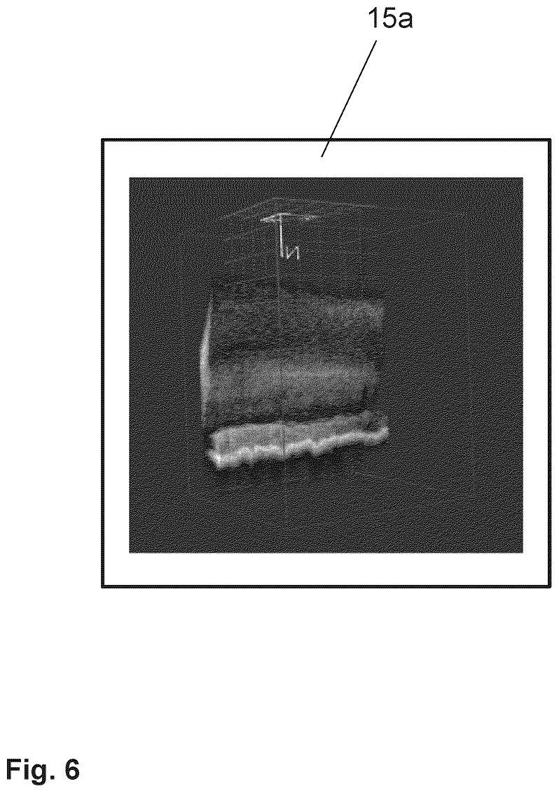

FIGS. 5, 6 show, on the basis of two virtual gonioscopy views, two visualizations of the same recording of an OCT volume, wherein in each case a specific detail from the iridocorneal angle is shown.

FIG. 1 shows a detailed sectional view of a human eye 1, in which the cornea 2 and iris 3 form a formation in the anterior eye chamber. This formation is referred to as the iridocorneal angle 4. Aqueous humor, which is shown by dashed lines here, can drain off through the iridocorneal angle 4. The so-called Schlemm's canal 5 extends in a ring shape in the scleral component of the iridocorneal angle 4, thus in the area of the sclera, and forms a collecting tube, namely a drain for the aqueous humor. In FIG. 1, the lens 6, the trabecular meshwork 7, the sclera 8, and the ciliary body 9 are shown.

FIG. 2 shows that a gonioscopic lens 10 is placed on the cornea 2. The structures in the iridocorneal angle 4 of the eye 1 cannot be seen directly without technical aids because of the refraction conditions at the cornea 2. In order to visualize these structures, a gonioscopic lens 10 is currently used, which directly contacts the cornea 2 in order to bypass the refraction at the front side thereof. Operations are currently carried out exclusively with the aid of these gonioscopic lenses 10.

However, the beam path 11 of the gonioscopic lens 10 causes a shadow of an instrument 12 to be thrown on the relevant structures Schlemm's canal 5 and trabecular meshwork 7 of the iridocorneal angle 4. In large part, the iridocorneal angle 4 itself lies in the shadow of the instrument 12.

FIG. 3, in contrast, shows how a three-dimensional volume 14 of a sample, namely the human eye 1, can be captured by means of optical coherence tomography (OCT) as a raw data set by means of a device 13 for performing gonioscopy, wherein a visualization unit 15 is provided, using which a virtual gonioscopy view 15a can be generated from the raw data set, which appears to be recorded from a virtual camera plane 16 in the interior of the sample, here the eye 1. The virtual gonioscopy view 15a is displayed on a monitor of the device 13.

The OCT beam path 17 of the device 13 does not cause a shadow of the instrument 12 to be thrown on the relevant structures Schlemm's canal 5 and trabecular meshwork 7 of the iridocorneal angle 4. Rather, the shadow of the instrument 12 is thrown on the iris 3, which is not relevant for an operation. In FIG. 4, a gonioscopic lens 10 is simply not used in comparison to FIG. 3, but rather a device 13, namely an OCT device.

The visualization unit 15 of the device 13 renders a recording of the three-dimensional volume 14, specifically handles it as a raw data set, and generates the virtual gonioscopy view 15a from the virtual camera plane 16 from this raw data set. The real camera plane 18 and the virtual camera plane 16 enclose an angle 19 from the range 80° to 140°.

FIG. 4 schematically shows that at least one volume 14 can be recorded using a detection unit 20 along an arc section, preferably over an angle range 23 of 10° to 120°. In FIG. 4, the pupil 21, the limbus 22, the circular angle range 23 for the recording, a virtual camera plane 16, and a camera center 24 are shown. Multiple volumes 14 can be recorded in chronological succession and captured as raw data sets. The arc section is part of a ring segment 25.

By means of the visualization unit 14, the external instrument 12 for the operative treatment of the eye 1 can be displayed transparent in the virtual gonioscopy view 15a or can be optically removed therefrom.

A tracking unit 26 is provided, using which the light for carrying out the optical coherence tomography on a volume 14 is trackable to a movement of the sample. An adjustment unit 27 is also provided, using which the virtual camera plane 16 can be determined and defined automatically on the basis of structures of the sample. The adjustment unit 27 is manually operable such that the position and orientation of the virtual camera plane 16 are settable in a user-controlled manner.

The volumes 14 are recordable at a repetition rate from the range 1 Hz to 1 kHz. The latency at which the virtual gonioscopy views 15a are displayable is less than 500 ms.

The device 13 described here performs a method for performing virtual gonioscopy, in which a gonioscopy lens 10 is not used, but rather the device 13 for performing optical coherence tomography. A device 13 of the above-described type is insofar used to perform the method.

The method is schematically described on the basis of FIGS. 3 to 6. The method comprises the following steps:
capturing a three-dimensional volume 14 of an eye 1 by means of optical coherence tomography as a raw data set from a real camera plane 18, in that the eye 1 is illuminated therefrom.
generating a virtual gonioscopy view 15a from the raw data set, wherein the virtual gonioscopy view 15a takes place from a virtual camera plane 16, which lies in the eye 1 and permits a top view of structures of the eye 1.
displaying structures of the iridocorneal angle 4 of the eye 1 in the virtual gonioscopy view 15a.

FIGS. 5 and 6 show two visualizations of the same OCT volume recording of a volume 14. In each case a detail from the iridocorneal angle 4 is accordingly visible. When observing the two visualizations, it is to be considered that sectional images precisely through the mechanical axis of the instrument 12 are seen. In the gonioscopic full face views, only a part of the view is concealed by the shadow of the instrument 12.

The structures comprise Schlemm's canal 5, the ciliary band, the scleral band, the trabecular meshwork 7, and/or Schwalbe's line.

A volume 14 is projected onto the virtual camera plane 16 such that the projected image is similar to a view through a gonioscopic lens 10 and thus forms the virtual gonioscopy view 15a.

The recording of the volume 14 takes place over an angle range 23 of 10° to 120° along the course of the limbus 22, as is schematically shown in FIG. 4.

The method is performed intraoperatively, in particular during glaucoma-surgery interventions.

### LIST OF REFERENCE SIGNS

1 eye
2 cornea
3 iris
4 iridocorneal angle
5 Schlemm's canal
6 lens
7 trabecular meshwork
8 sclera
9 ciliary body
10 gonioscopic lens
11 beam path of the gonioscopic lens
12 instrument
13 device
14 volume of 1
15 visualization unit
15a virtual gonioscopy view
16 virtual camera plane
17 OCT beam path 17 of the device
18 real camera plane
19 angle
20 detection unit
21 pupil
22 limbus
23 angle range
24 camera center
25 ring segment
26 tracking unit
27 adjustment unit

1. A device for performing gonioscopy, using which a three-dimensional volume of a sample can be captured by means of optical coherence tomography as a raw data set, characterized in that a visualization unit is provided, using which a virtual gonioscopy view can be generated from the raw data set, which appears to be recorded from a virtual camera plane in the interior of the sample.

2. The device as claimed in claim 1, wherein the visualization unit renders a recording of the three-dimensional volume, specifically handles it as a raw data set and generates from this raw data set the virtual gonioscopy view from the virtual camera plane.

3. The device as claimed in claim 1, wherein a real camera plane and the virtual camera plane enclose an angle from the range 80° to 140°, preferably in that the two camera planes are oriented orthogonally to one another.

4. The device as claimed in claim 1, wherein at least one volume along an arc section, preferably over an angle range of 10° to 120°, is recordable using a detection unit.

5. The device as claimed in claim 1, wherein multiple volumes can be recorded in chronological succession and can be captured as raw data sets.

6. The device as claimed in claim 4, wherein the arc section is part of a ring segment or part of a spiral, in particular an elliptical spiral.

7. The device as claimed in claim 1, wherein by means of the visualization unit, an external instrument for the operative treatment of the sample is displayable transparent in the virtual gonioscopy view or is optically removable therefrom.

8. The device as claimed in claim 1, wherein a tracking unit is provided, using which the light for performing the optical coherence tomography is trackable to a volume of a movement of the sample.

9. The device as claimed in claim 1, wherein an adjustment unit is provided, using which the virtual camera plane can be automatically determined and defined on the basis of structures of the sample, and/or in that the adjustment unit is manually operable such that the position and orientation of the virtual camera plane are settable in a user-controlled manner.

10. The device as claimed in claim 1, wherein the volumes are recordable at a repetition rate from the range 1 Hz to 1 kHz, and/or in that the latency with which the virtual gonioscopy views are displayable is less than 500 ms.

11. A method for performing virtual gonioscopy, in which a gonioscopic lens is not used, but rather a device for performing optical coherence tomography, in particular as claimed in claim 1, comprising the following steps:
capturing a three-dimensional volume of an eye by means of optical coherence tomography as a raw data set from a real camera plane, in that the eye is illuminated therefrom,
generating a virtual gonioscopy view from the raw data set, wherein the virtual gonioscopy view takes place from a virtual camera plane, which lies in the eye and permits a top view of structures of the eye,
displaying structures of the iridocorneal angle of the eye in the virtual gonioscopy view.

12. The method as claimed in claim 11, wherein the structures comprise Schlemm's canal, ciliary band, scleral band, trabecular meshwork, and/or Schwalbe's line.

13. The method as claimed in claim 11, wherein the recording of the volume takes place over an angle range of 10° to 120° along the course of the limbus.

14. The method as claimed in claim 11, wherein a volume is projected on the virtual camera plane such that the projected image is similar to a view through a gonioscopic lens and thus forms the virtual gonioscopy view.

15. The method as claimed in claim 11, wherein the method is carried out intraoperatively, in particular during glaucoma-surgery interventions.
